(19) United States
(12) Patent Application Publication
(10) Pub. No.: US 2015/0313752 A1
(43) Pub. Date: Nov. 5, 2015

(54) CONDOM SYSTEM AND METHOD FOR MAKING

(71) Applicant: Max ABADI, Bogota (CO)

(72) Inventor: Max ABADI, Bogota (CO)

(21) Appl. No.: 14/266,541

(22) Filed: Apr. 30, 2014

(51) Int. Cl. A61F 6/04

(52) U.S. Cl. CPC A61F 6/04

(57) ABSTRACT

A prophylactic apparatus includes an elongated, substantially tubular article made of film material. The elongated tubular article has a closed, sealed end, and an open end. The open end and the elongated tubular portion are dimensioned to fit over a flaccid penis. The prophylactic apparatus is also dimensioned so that at the penis achieves a turgid state, the prophylactic apparatus will fit snugly and produce a seal between the prophylactic apparatus and the users penis. The method of placing the prophylactic apparatus is also disclosed.

### TECHNICAL FIELD

Various embodiments described herein relate to a condom system and method for making the condom.

### BACKGROUND

Condoms have been a highly favored alternative among men who wish to take part in sexual activities but want to avoid sexually transmitted diseases or avoid pregnancy in their partner. A wide variety of condoms or prophylactic items are constructed in the form of a protective sheath of thin, flexible material. Commonly, condoms are supplied in a rolled up configuration. Most condoms are currently packaged by rolling them and placing them in a plastic wrapper, which helps protect the condom from physical and chemical damage, including that caused by dryness and ultraviolet rays. As packaged, the condom maintains a substantially circular shape. The package is generally square-shaped and includes a substantially circular chamber.

Applying the condom requires opening the package, removing the condom from the chamber, correctly positioning the condom over the erect penis and unrolling the condom over the length of the penis. This generally requires an inconvenient break while the condom is applied.

In some instances, condoms may be coated with, or otherwise immersed in, one or more compounds providing a lubricant, a spermicide, or a bactericide, all of which render the condom difficult, or at least uncomfortable, to handle during application. In addition, these one or more compounds can make the condom uncomfortable to wear over an extended period of time. Furthermore, the various compounds can stain undergarments or the like. In addition, removal may be difficult, or uncomfortable, and may require careful handling to maintain aseptic conditions, or at least a modicum of cleanliness.

### BRIEF DESCRIPTION OF THE DRAWINGS

The invention is pointed out with particularity in the appended claims. However, a more complete understanding of the present invention may be derived by referring to the detailed description when considered in connection with the figures, wherein like reference numbers refer to similar items throughout the figures and:

FIG. 1 is a perspective view of the card package as a user removes a condom or prophylactic apparatus from the cavity in the card package, according to an example embodiment.

FIG. 2 is a perspective view of a prophylactic apparatus formed from a film material, according to an example embodiment.

FIG. 3 is a perspective view of a prophylactic apparatus formed from film according to an example embodiment.

FIG. 3 is a perspective view of a prophylactic apparatus as formed from film, being placed on a mandrel for rolling the prophylactic apparatus, according to an example embodiment.

FIG. 4 is a perspective view of a prophylactic apparatus after being placed on the mandrel, according to an example embodiment.

FIG. 5 is a perspective view of a prophylactic apparatus pulled halfway up the mandrel, according to an example embodiment.

FIG. 6 is a perspective view of a prophylactic apparatus as the tails of the strip of cloth are initially pulled to roll the prophylactic apparatus, according to an example embodiment.

FIG. 7 is a perspective view of a prophylactic apparatus being rolled up after being pulled halfway up upon itself, according to an example embodiment.

FIG. 8 is a perspective view of a prophylactic apparatus at the point where the prophylactic apparatus is fully rolled up, according to an example embodiment.

FIG. 9 is a perspective view of a prophylactic apparatus as rolled in FIGS. 5 through 8, according to an example embodiment.

FIG. 10 is a perspective view of a prophylactic apparatus after it has been partially unrolled, according to an example embodiment.

FIG. 11 is a flow chart of a method of applying a prophylactic apparatus, according to an example embodiment.

FIG. 12 is an instruction set that accompanies the prophylactic apparatus or condom, according to an example embodiment.

FIG. 13 is a perspective view of the card package as a user removes a portion of the cover from a cavity in the card package, according to an example embodiment.

### DETAILED DESCRIPTION

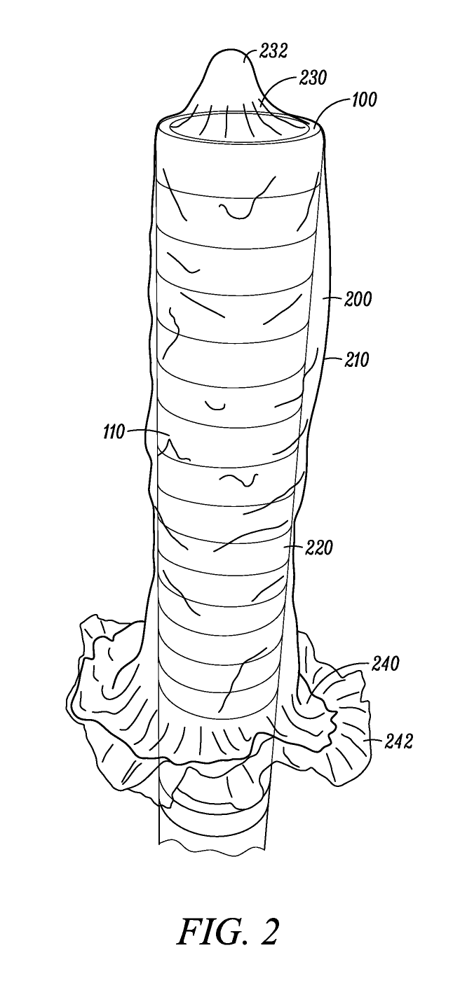

FIG. 2 is a perspective view of a prophylactic apparatus 200 after being formed from a film material 210, according to an example embodiment. The film 210 is shaped deforming the film material to the prophylactic apparatus 200.

After being formed from the film material 210, the prophylactic apparatus 200 is placed on a mandrel. The mandrel, shown in FIG. 2, has a cylindrical body with a diameter dimension which is less than the diameter of the cylindrical body or tubular portion 110 of the mandrel 100. The mandrel shown in FIG. 2 holds the prophylactic apparatus 200 until further processed. As formed, the prophylactic apparatus 200 includes a tubular portion 220, closed end 230 with a receptacle or fluid reservoir 232 therein, and with an open end 240. As shown, the open end 240 includes a skirt 242 which flares out from the open end. The skirt 242 is attached to the open end 240. The skirt 242 can be trimmed to remove excess material. In some embodiments the skirt 242 can be trimmed to leave one or more tabs. In other embodiments the skirt 242 is trimmed to eliminate excess material around the periphery of the skirt. As shown in FIG. 2, the film material 210 is not fully stretched out and has a corrugated appearance. In other words the film material 210 is not fully stretched, as shown in this figure. The prophylactic apparatus 200 is placed on the mandrel to be folded and rolled before placed in a package.

Film material 210 can be made of polyvinylidene chloride (PVdC). PVdC has better barrier properties than the more-permeable LDPE, even though LDPE is substantially cheaper and easier to make. LDPE is a lower quality film because PVdC has a significantly lower water vapor transmission rate (WVTR) than LDPE.

The PVC-based films contain plasticizers, most often bis (2-ethylhexyl) adipate (DEHA), phthalates or dibutyl phthalate (DBP) or bis (2-ethylhexyl) phthalate (DEHP)). Some countries prohibit these plasticizers since they migrate out of the thin plastic film. Polymerized plasticizers replaced DEHP, largely eliminating migration of the plasticizer out of the film.

Natural polymers of LDPE and PVdC are insufficiently adhesive on their own, and they do not adhere to themselves. To achieve the desired adhesiveness, certain polymers with lower molecular weight have to be added; the most common two are polyisobutene (PIB), and poly[ethylene-vinylacetate] (EVA) copolymer. Their chains readily interact with each other and their lower molecular weight makes them more mobile within the host polymer matrix.

The material of the prophylactic apparatus 200 can be a film resin. The types of materials that could be used are many and varied. Should be noted that the traditional material of latex rubber or lamb intestine or polyurethane is not used.

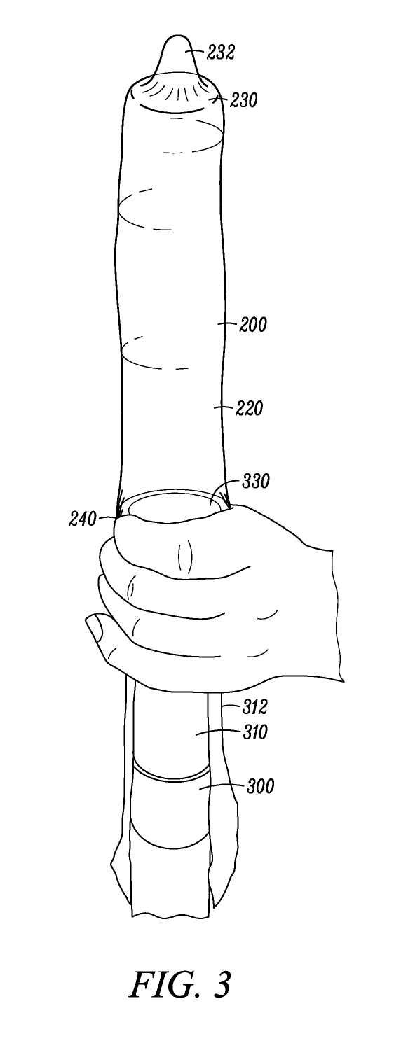

FIG. 3 is a perspective view of a prophylactic apparatus 200 as formed from film, being placed on a mandrel for rolling the prophylactic apparatus 200, according to an example embodiment. The mandrel 300 as a tubular portion 310 which is slightly larger in diameter than the tubular portion shown in FIG. 2 and slightly smaller than the tubular portion 110 of the mandrel 100. A strip of cloth 312 is placed over the end 330 of the mandrel 300. The strip of cloth 312 is much longer than the mandrel 300. The strip of cloth 312 has sufficient length to pass over one elongated side of the mandrel 310, the end 330 and over the other elongated side of the mandrel 310. The strip of cloth 312 is used to enable or aid in rolling the prophylactic apparatus 200. After placing the strip of cloth 312 over the end of the mandrel 310, the prophylactic device is placed onto the mandrel. As shown in FIG. 3, the prophylactic device is placed onto the mandrel by hand. It is contemplated that in other embodiments, the prophylactic device can be placed onto the mandrel using a robot or other mechanical device.

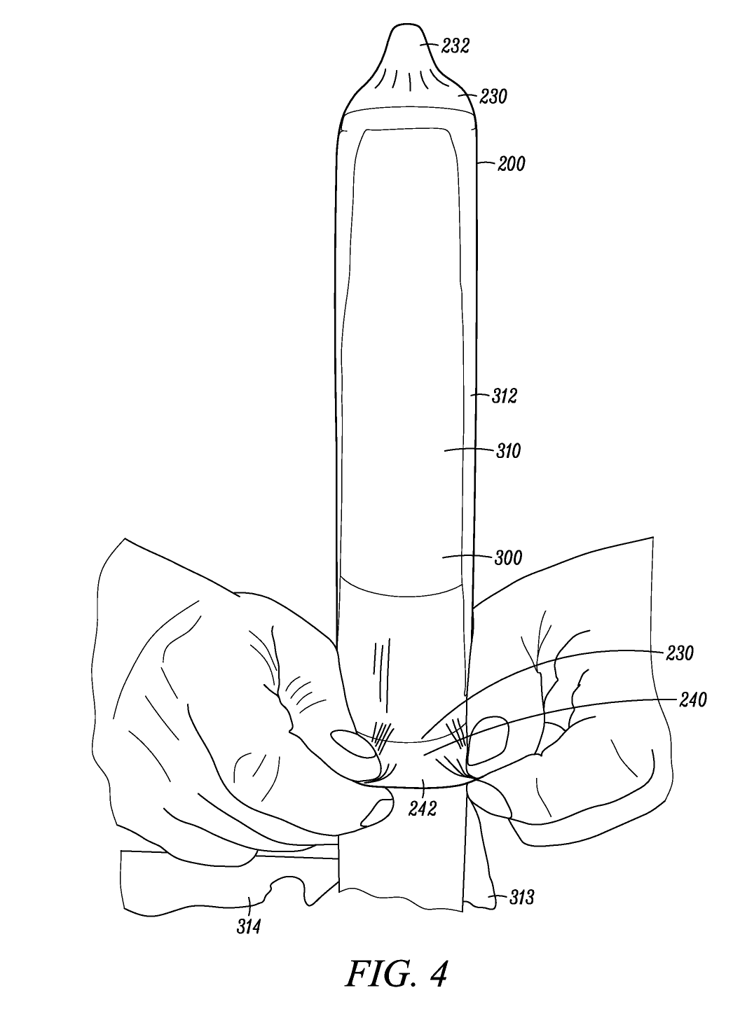

FIG. 4 is a perspective view of a prophylactic apparatus 200 after being placed on the mandrel 300, according to an example embodiment. The prophylactic apparatus 200 is fully deployed on the mandrel 300. The strip of cloth 312 has a first tale 313 in a second tale 314 which extends beyond the open and 230 and the skirt 240 of the prophylactic apparatus. In one embodiment, the tales 313 and 314 of the strip of fabric 312 can be pulled upwardly in order to roll the bottom portion of the prophylactic apparatus 200 toward the closed end 230. In this way the skirt 242 the closed end 240 and a majority of the tubular portion 220 is rolled onto itself toward the closed end 230. The prophylactic apparatus 200, as rolled, can then be packaged.

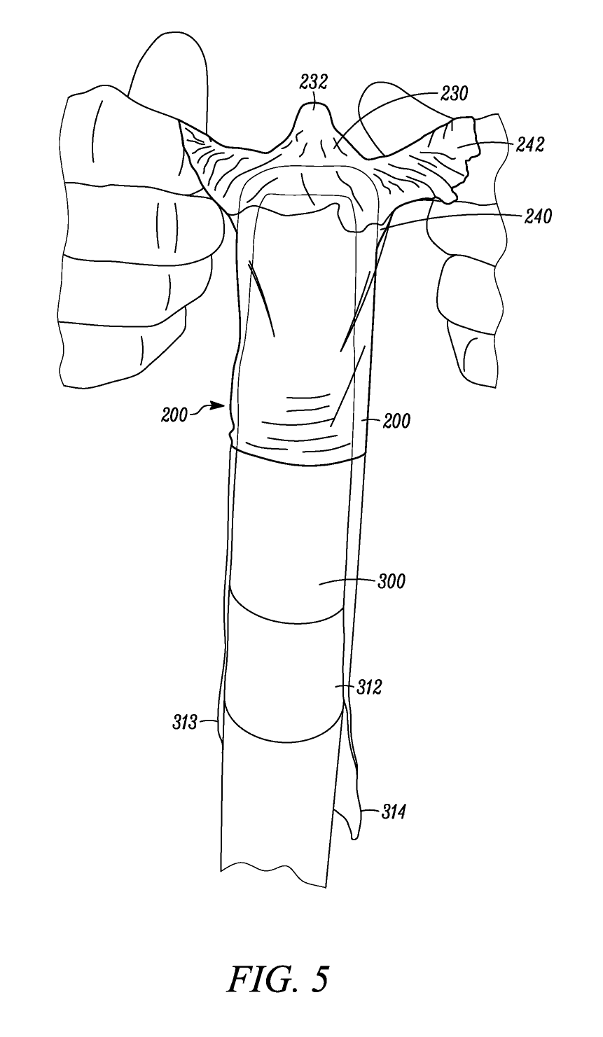

FIG. 5 is a perspective view of a prophylactic apparatus 200 pulled halfway up the mandrel 300, according to an example embodiment. This particular embodiment, the prophylactic apparatus 200 is pulled onto itself. In other words, the skirt 242, the open end 240 and the lower portion of the tubular portion 230 are pulled upward on the mandrel so that the lower portion lays on top of the upper portion of the tubular portion 230. As shown in FIG. 5, the tubular portion 230 is forming two layers on the upper portion of the mandrel 300. The tubular portion 230 is folded onto itself or pulled up onto itself. This leaves the mandrel 300 with the prophylactic apparatus extending about halfway down the mandrel 300. The top portion of the tubular portion 230 is below the bottom portion of the tubular portion 230, as viewed in FIG. 5. The strip of cloth 312 and specifically the tails 313 and 314 are pulled upwardly to roll the prophylactic apparatus or condom 200.

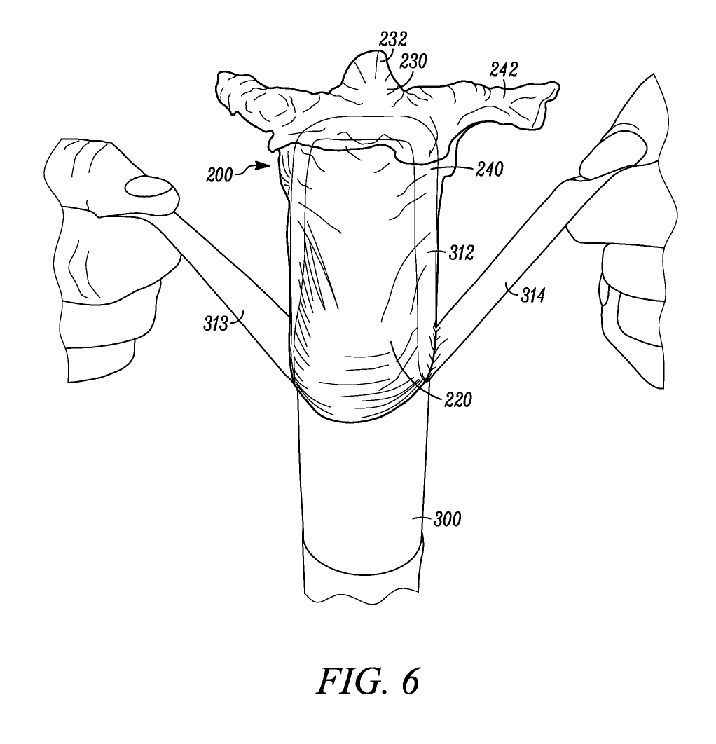

FIG. 6 is a perspective view of a prophylactic apparatus 200 as the tails 313, 314 of the strip of cloth 312 are initially pulled to roll the prophylactic apparatus 200, according to an example embodiment. The tails 313, 314 of the strip of cloth 312 are grasped and an upwardly acting force is applied onto the tails 313, 314. In this embodiment, this starts the rolling of the prophylactic apparatus 200. The roll includes two layers of plastic film 200. The roll also includes two layers of the tubular portion 220 of the prophylactic apparatus 200.

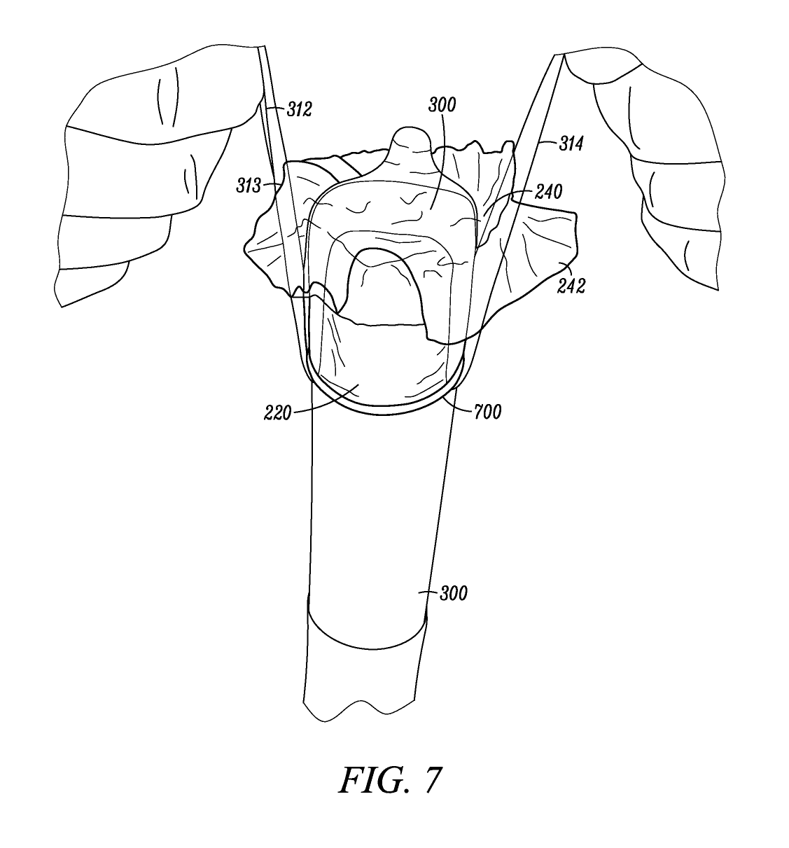

FIG. 7 is a perspective view of a prophylactic apparatus 200 being rolled up after being pulled halfway up, according to an example embodiment. The tails 313, 314 of the cloth strip 312 are further pulled up and a roll 700 is formed as the two layers of the tubular portion 220 of the prophylactic apparatus 200 roll onto one another. An upward force continues to be applied as the roll 700 becomes more closely positioned with respect to the top and 330 of the mandrel 300.

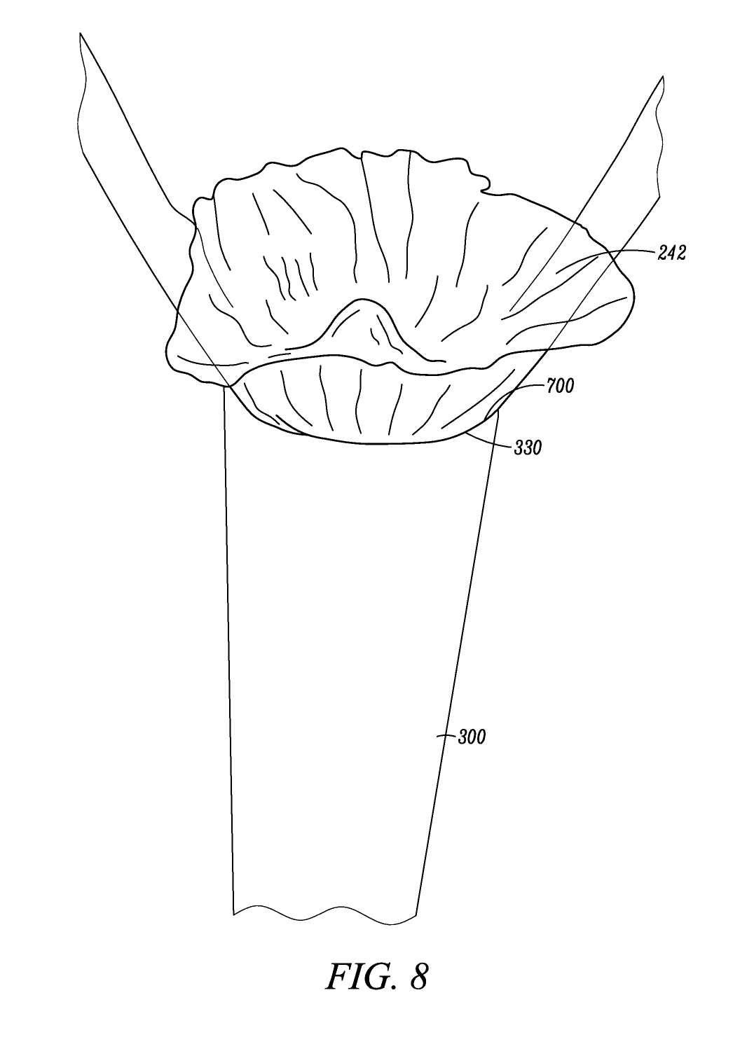

FIG. 8 is a perspective view of a prophylactic apparatus 200 at the point where the prophylactic apparatus 200 is fully rolled up, according to an example embodiment. The roll 700 (shown in FIG. 7) eventually reaches the top portion 330 of the mandrel 300. The roll 700 is eventually pulled off the main cylindrical portion of the mandrel 300. FIG. 8 shows the prophylactic apparatus as rolled up just as it is about to be taken away from the mandrel 300. The tails 313, 314 of the strip of cloth 312 are still being pulled upwardly.

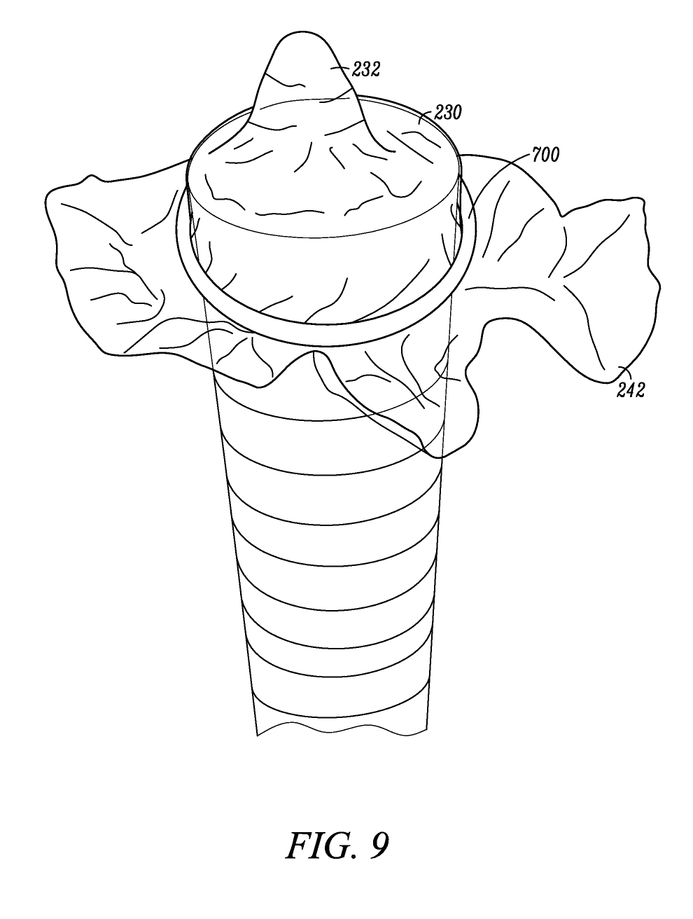

FIG. 9 is a perspective view of a prophylactic apparatus as rolled in FIGS. 5 through 8, according to an example embodiment. The prophylactic apparatus 200 is rolled is ready for packaging. Prophylactic apparatus includes a roll 700 that encompasses or contains most of the tubular portion 220 of the prophylactic apparatus 200. Prophylactic apparatus shows the skirt 242 extending out beyond the roll 700 and the closed end portion 230 positioned within the circumference of the roll 700. One advantage of this particular configuration is that the prophylactic apparatus can be unrolled by pulling on either the skirt 242 or the closed and 230 of the prophylactic apparatus 200. FIG. 9 shows the prophylactic apparatus 200 on a smaller diameter mandrel 900. The smaller diameter mandrel 900 represents a flaccid penis. Another advantage of this prophylactic apparatus is that it can be deployed upon a flaccid penis well before the prophylactic apparatus 200 needs to be used. The skirt 242 of the prophylactic device 200 can be pulled or pulled downward to deploy the prophylactic apparatus 200.

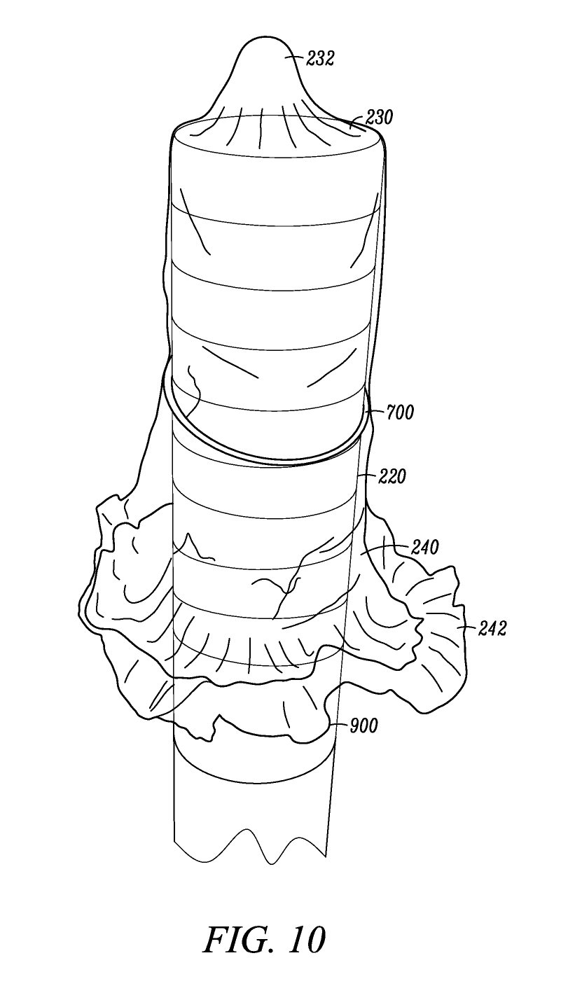

FIG. 10 is a perspective view of a prophylactic apparatus 200 after it has been partially unrolled, according to an example embodiment. Prophylactic apparatus 200 is partially deployed or partially unrolled. The roll 700 goes down the mandrel 900 as the prophylactic device is unrolled. The role 700 still includes a double layer of a portion of the tubular portion 220 of the prophylactic apparatus 200. The skirt 242 of the prophylactic device 200 continues to be pulled until the role 700 disappears. At this point the prophylactic apparatus 200 is fully deployed or fully unrolled.

The prophylactic apparatus 200 as shown and discussed above has the advantage that can be deployed onto a flaccid penis. The prophylactic apparatus can be deployed well before use. Therefore if the user anticipates that he may become sexually active in the next short timeframe, he can deploy this prophylactic apparatus onto a flaccid penis and wear it until used at a later time. As the penis becomes turgid the penis also becomes larger in diameter. The prophylactic apparatus 200 is dimensioned so that it fits loosely over a flaccid penis and fits tightly over a turgid penis. The material or thin plastic film 210 of the prophylactic apparatus is capable of stretching and adhering to the turgid penis. This forms a seal during sexual activity. The thin plastic film 210 is also sufficiently strong so as not fail during sexual activity.

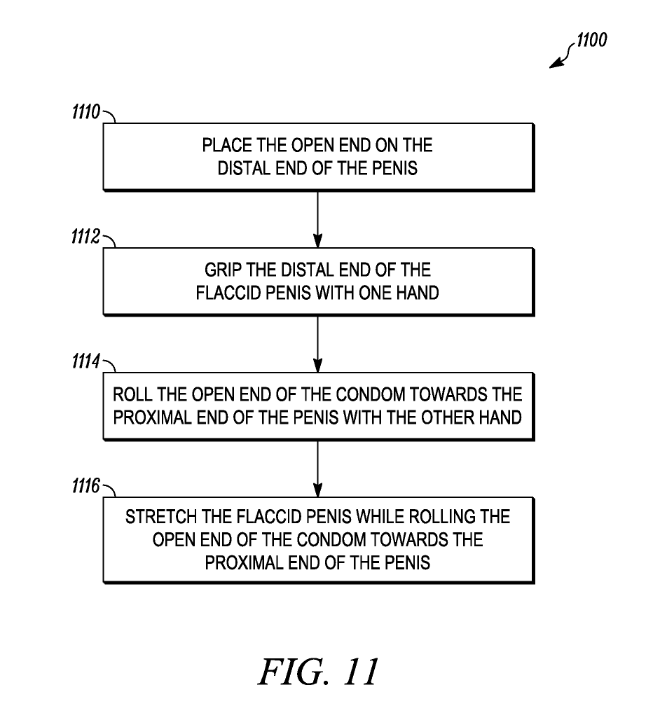

FIG. 11 is a flow chart of a method 1100 of applying a prophylactic apparatus, according to an example embodiment. The method 1100 of applying a condom to a flaccid penis where the condom made of film material and has a substantially tubular shape with a closed end and an open end includes placing the open end on the distal end of the penis 1110, gripping the distal end of the flaccid penis with one hand 1112, and rolling the open end of the condom toward the proximal end of the penis with the other hand 1114. The method 1100, in some embodiments, further includes stretching the flaccid penis while rolling the open end of the condom toward the proximal end of the penis 1116. Of course the prophylactic apparatus 200 must be removed from a package before the prophylactic apparatus for condom 200 is applied.

Since the condom is made to be deployed on a flaccid penis, the user is able to apply the condom well before use. The user can place the condom on the penis at home or before going out. In this fashion, the condom will be in place for later use. The condom is dimensioned so that it fits snuggly enough on the flaccid penis so that it will not inadvertently fall off or become dislodged from the user's penis before use. The film material, from which the condom is formed, is adapted to stretch to accommodate any increase in size as the penis becomes erect. When erect, the film material stretches to form a seal to prevent fluid leakage. The film material stretches rather than fails. The film material is of sufficient strength so that it will not fail during intercourse.

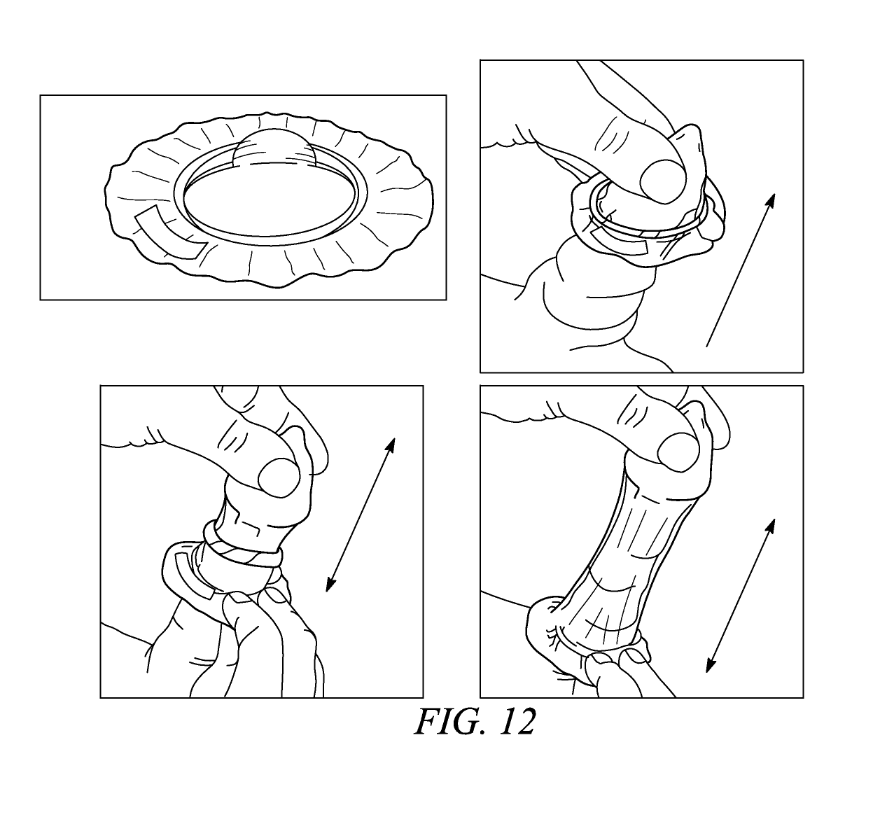

FIG. 12 is an instruction set that accompanies the prophylactic device or condom 200, according to an example embodiment. The instruction set sets forth advantages as well as step-by-step instructions for the user. Among the advantages mentioned in the instruction set is that the condom or prophylactic apparatus 200 can be applied up to two hours before foreplay, without direction. In addition there is no interruption in sexual activity needed while the person places the condom on the erect penis. Furthermore the material is hypoallergenic genic, transparent, extra soft and odorless and will cling to the penis making it ultra sensitive. The base flange or skirt 242 provides for more protection. Instructions also show that the prophylactic apparatus 200 can be provided with a label and that the label is to be placed upwards and visible during application of the prophylactic apparatus 200. The instructions also set forth that the prophylactic apparatus 200 is placed on the head of the penis and then the head of the penis is placed into the condom an extended onto the penis. In addition, the head of the penis is held, and the base flange or skirt 242 is pulled gently toward the base of the penis. The condom is then pulled to the base of the penis until the condom feels smooth. Air is then removed from the condom.

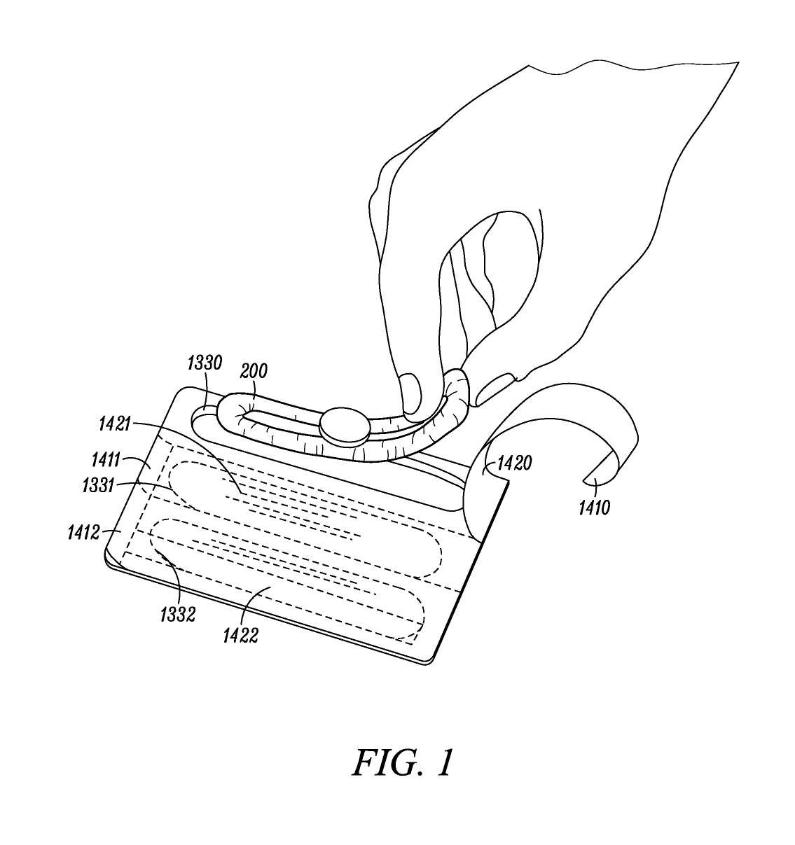

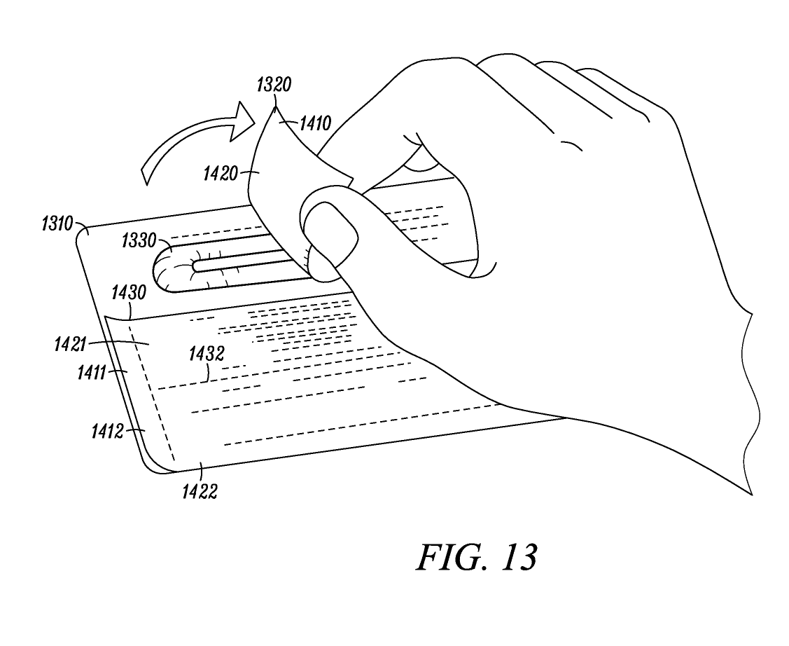

FIG. 13 is a perspective view of the card package as a user removes a portion of the cover 1320 from a cavity 1330 in the card package 1300, according to an example embodiment. FIG. 1 is a perspective view of the card package 1300 as a user removes a condom or prophylactic apparatus 200 from the cavity 1330 in the card package, according to an example embodiment. As shown in FIGS. 13 and 1, the card body 1300 includes a cavity 1330 therein. The cavity 1330 is substantially oblong or oval-shaped. In some example embodiments, there are multiple cavities. FIG. 4 shows an embodiment that includes multiple cavities 1330, 1331, 1332. The cavities 1321, 1322 are shown in phantom. A condom, such as condom 200, fits within the cavity 1330. The condom 200 is pressed into a oval or hour-glass shaped. The cavity 1330 or cavities 1330, 1331, 1332 in the card body 1310 are formed by using a die which, by pressing the plastic also cuts the plastic, and leaves the cavity 1330 or multiple cavities 1330, 1331, 1332. In another embodiment, the package card 1300 is formed by injection molding. Injection molding permits a package card 1300 to be produced with a cavity or cavities already incorporated. The cavities 1330, 1331, 1332 are sized and shaped to house a condom, such as condom or prophylactic apparatus 200. Of course, the size and shape of the cavities 1330, 1331, 1332 can vary depending on the size and shape of the condom, such as condom 200, which are housed within the cavities 1330, 1331, 1332.

The number of cavities can also vary depending on the number of condoms which are to be included in the package card 1300. In one example embodiment, the package card 100 has a single cavity 130. In another example embodiment, the package card 100 has a plurality of cavities 1330, 1331, 1332. As shown, the cover 1320 can include perforations 1430, 1432. The perforations provide for a weak line along which the cover can tear away. The perforations 1430, 1432 separate the cover 1320 into separate cover portions 1420, 1421, 1422. Cover portion 1420 fits over and seals cavity 1300. Cover portion 1421 fits over and seals cavity 1331. Cover portion 1422 fits over and seals cavity 1332. As shown in FIGS. 13 and 1, the cover portions 1420, 1421 and 1422 include a tab 1410, 1411, 1412, respectively. The tab 1410, 1411, 1412 is a free end which is free of adhesive and of a size so that it can be lifted and pulled using a person's digits.

FIG. 1 is a perspective view of the card package as a user further removes a cover 1420 over the cavity 1330 and reveals a condom or prophylactic apparatus within the cavity 1330 in the card package, according to an example embodiment.

A skirt, in one embodiment, is formed at the base of the condom. The skirt is attached to the open end of the condom. In some embodiments, the excess film material is trimmed. This can be trimming the skirt to make it a certain length or can be trimming the skirt to form one or more tabs attached to the base of the condom. The method also includes rolling the condom, and packaging the rolled condom. In one embodiment, rolling the condom apparatus includes pulling the open end of the tubular portion of the condom to a position proximate the closed end, and rolling the condom from the tubular portion between the closed and open ends of the condom to a position near the closed end. The condom can also be rolled from the open end toward the closed end, and placed in a package.

A prophylactic apparatus includes an elongated, substantially tubular article made of film material. The elongated tubular article has a closed, sealed end, and an open end. The open end and the elongated tubular portion are dimensioned to fit over a flaccid penis. The prophylactic apparatus also has a skirt portion attached the open end. The skirt flares out beyond a periphery of the open end. The skirt can be gripped to deploy the prophylactic apparatus. The prophylactic apparatus is dimensioned to stretch so that the tubular portion stretches when the flaccid penis becomes erect. In other words, the film material deforms to accommodate the size increase between a flaccid penis and an erect penis. The film, in one embodiment, deforms plastically. In another embodiment, the film deforms elastically. In either embodiment, the film engages the erect penis to form a seal about the penis. In one embodiment, the tubular portion is rolled to a position near the closed end. At least a portion of the skirt portion is exposed when the tubular portion is rolled. The prophylactic apparatus as rolled up is placed within a package. In one embodiment, the skirt is initially pulled up to place a bottom portion of the tubular portion onto a top portion of the tubular portion and rolled to a position near the closed end. A substantial portion of the tubular portion of the prophylactic apparatus is double layered when the prophylactic apparatus is rolled up in this fashion. When rolled in this fashion, the prophylactic apparatus can be unrolled from the closed end or by exerting a force on the skirt portion. The rolled prophylactic apparatus is packaged.

This has been a detailed description of some exemplary embodiments of the invention(s) contained within the disclosed subject matter. Such invention(s) may be referred to, individually and/or collectively, herein by the term “invention” merely for convenience and without intending to limit the scope of this application to any single invention or inventive concept if more than one is in fact disclosed. The detailed description refers to the accompanying drawings that form a part hereof and which shows by way of illustration, but not of limitation, some specific embodiments of the invention, including a preferred embodiment. These embodiments are described in sufficient detail to enable those of ordinary skill in the art to understand and implement the inventive subject matter. Other embodiments may be utilized and changes may be made without departing from the scope of the inventive subject matter. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed:

1. A prophylactic apparatus comprising:
an elongated substantially tubular article made of film material, the elongated tubular article having:
a closed, sealed end; and
an open end, the open end and the elongated tubular portion dimensioned to fit over a flaccid penis;
a skirt portion attached the open end, the skirt flaring out beyond a periphery of the open end, the condom dimensioned to fit over a flaccid penis without falling off.

2. The prophylactic apparatus of claim 1 wherein the tubular portion is dimensioned to stretch when the flaccid penis becomes erect.

3. The prophylactic apparatus of claim 1 wherein the film material deforms to accommodate the size increase between a flaccid penis and an erect penis.

4. The prophylactic apparatus of claim 3 wherein the film deforms plastically.

5. The prophylactic apparatus of claim 3 wherein the film deforms elastically.

6. The prophylactic apparatus of claim 1 wherein the tubular portion is rolled to a position near the closed end.

7. The prophylactic apparatus of claim 6 wherein at least a portion of the skirt portion is exposed when the tubular portion is rolled.

8. The prophylactic apparatus of claim 6 further comprising a package, the prophylactic apparatus as rolled up placed within the package.

9. The prophylactic apparatus of claim 1 wherein the skirt is initially pulled up to place a bottom portion of the tubular portion onto a top portion of the tubular portion and rolled to a position near the closed end.

10. The prophylactic apparatus of claim 9 wherein the rolled prophylactic apparatus can be unrolled from the closed end or by exerting a force on the skirt portion.

11. The prophylactic apparatus of claim 9 further comprising a package, wherein the rolled prophylactic apparatus is packaged.

12. A method for forming a condom assembly comprising:
forming a condom by plastically deforming a film material;
rolling the condom which includes;
pulling the open end of the tubular portion of the condom to a position proximate the closed end; and
rolling the condom from the tubular portion between the closed and open ends of the condom to a position near the closed end; and packaging the rolled condom.

13. The method of claim 12 further wherein rolling the condom includes rolling the tubular portion from the open end toward the closed end.

14. A method of applying a condom to a flaccid penis, the condom made of a film material, substantially tubular in shape and having a closed end and an open end, comprising:
placing the open end on the distal end of the penis;
gripping the distal end of the flaccid penis with one hand; and
rolling the open end of the condom toward the proximal end of the penis with the other hand, the condom dimensioned to fit the flaccid penis.

15. The method of claim 14 further comprising stretching the flaccid penis while rolling the open end of the condom toward the proximal end of the penis.

16. The method of claim 14 further comprising wearing the condom on the flaccid penis for at least one hour before using the condom.

17. The method of claim 14 wherein the condom stretches to accommodate an increase in size resulting in the penis going to an erect state from a flaccid state.

18. The method of claim 14 further comprising covering the flaccid penis and applied condom.

19. The method of claim 14 wherein the condom stretches in response to an erection and wherein the condom is used without substantial adjustment after placing the condom on the flaccid penis.
